(19) United States
(12) Patent Application Publication
(10) Pub. No.: US 2025/0125007 A1
(43) Pub. Date: Apr. 17, 2025

(54) COMPUTING SYSTEM FOR CUSTOMIZING AND DEFINING PARAMETERS OF A MEDICAL-BASED ANALYSIS SYSTEM

(71) Applicant: Epiveris LLC, Steamboat Springs, CO (US)

(72) Inventor: Sonja S. Nakasian, Steamboat Springs, CO (US)

(21) Appl. No.: 18/914,344

(22) Filed: Oct. 14, 2024

Related U.S. Application Data
(60) Provisional application No. 63/590,159, filed on Oct. 13, 2023.

(51) Int. Cl. G16B 20/00; G06F 40/20; G16B 40/20; G16H 10/60; G16H 50/20

(52) U.S. Cl. CPC G16B 20/00; G06F 40/20; G16B 40/20; G16H 10/60; G16H 50/20

(57) ABSTRACT

A system includes a processor and a nontransitory computer-readable medium comprising instructions that are executable by the processor. The instructions include receiving data from at least one health database, performing a large language model (LLM) routine to transform the data into structured data, generating a phenotype routine by selectively modifying at least one parameter of a selected template phenotype routine of the plurality of template phenotype routines, analyzing the structured data based on the phenotype routine to generate an output that defines a relationship model associated with the structured data, and transmitting a command to a user interface to generate a display corresponding to the output.

### CROSS-REFERENCE TO RELATED APPLICATION

This application claims priority to U.S. provisional application No. 63/590,159 filed on Oct. 13, 2023. The disclosure of the above application is incorporated herein by reference.

### FIELD

The present disclosure relates to systems and methods for customizing and defining parameters of a medical-based analysis system.

### BACKGROUND

The statements in this section merely provide background information related to the present disclosure and may not constitute prior art.

Clinical and observational research studies may evaluate the effectiveness, safety, and impact of healthcare interventions and treatments. The data employed by the clinical and observational research studies may originate from different sources and have varying syntactic standards. As such, the processes of parsing through large amounts of data to select the appropriate data, identify relationships among the selected data, and standardizing the selected data for further analysis are resource-intensive and time-intensive processes.

### SUMMARY

This section provides a general summary of the disclosure and is not a comprehensive disclosure of its full scope or all of its features.

The present disclosure provides a system including a processor, a plurality of health databases, where each of the plurality of health databases has a respective syntactic standard, a phenotype routine database that stores a plurality of template phenotype routines, and a nontransitory computer-readable medium including instructions that are executable by the processor. The instructions include receiving data from at least one health database of the plurality of health databases, performing a large language model (LLM) routine to transform the data into structured data, generating a phenotype routine by selectively modifying at least one parameter of a selected template phenotype routine of the plurality of template phenotype routines, analyzing the structured data based on the phenotype routine to generate an output that defines a relationship model associated with the structured data, and transmitting a command to a user interface to generate a display corresponding to the output.

In some embodiments, the LLM routine is one of a generative pretrained transformer routine or a natural language processing routine.

In some embodiments, the instructions further include: generating a directed acyclic graph based on the structured data and at least one causal relationship associated with the structured data, and transforming the directed acyclic graph into test data, where the test data and the structured data have a same syntax, where analyzing the structured data based on the phenotype routine to generate an output that defines a relationship model associated with the structured data further includes analyzing the test data based on the phenotype routine.

In some embodiments, the instructions further include: selecting a first template phenotype routine of the plurality of template phenotype routines based on the structured data, and determining at least one performance metric of the first template phenotype routine, where the at least one performance metric of the first template phenotype routine includes at least one of a positive predictive value (PPV), a negative predictive value (NPV), a sensitivity value, and a specificity value of the first template phenotype routine.

In some embodiments, the instructions further include designating the first template phenotype routine as the selected phenotype routine when the at least one performance metric of the first template phenotype routine satisfies a model selection criteria.

In some embodiments, the instructions further include: selecting a second template phenotype routine of the plurality of template phenotype routines when the at least one performance metric of the first template phenotype routine does not satisfy a model selection criteria, determining at least one performance metric of the second template phenotype routine, where the at least one performance metric of the second template phenotype routine includes at least one of a positive predictive value (PPV), a negative predictive value (NPV), a sensitivity value, and a specificity value of the second template phenotype routine, and designating the second template phenotype routine as the selected phenotype routine when the at least one performance metric of the second template phenotype routine satisfies the model selection criteria.

In some embodiments, the instructions further include determining at least one performance metric of the selected template phenotype routine, the at least one parameter includes a number of dimensions of the selected template phenotype routine, and the instructions for generating the phenotype routine by selectively modifying the at least one parameter of the selected template phenotype routine further include iteratively reducing the number of dimensions of the selected template phenotype routine until the at least one performance metric satisfies an analytic threshold.

In some embodiments, the instructions further include determining at least one performance metric of the selected template phenotype routine, the at least one parameter includes at least one cluster characteristic of the selected template phenotype routine, and the instructions for generating the phenotype routine by selectively modifying the at least one parameter of the selected template phenotype routine further include iteratively modifying the at least one cluster characteristic of the selected template phenotype routine until the at least one performance metric satisfies an analytic threshold.

In some embodiments, the at least one cluster characteristic includes at least one of a number of dimensions of the selected template phenotype routine, a number of clusters of the selected template phenotype routine, and a distance metric of the selected template phenotype routine.

In some embodiments, the instructions further include determining at least one performance metric of the selected template phenotype routine, the at least one parameter comprises a reinforcement learning model parameter of the selected template phenotype routine, and the instructions for generating the phenotype routine by selectively modifying the at least one parameter of the selected template phenotype routine further include iteratively modifying the reinforcement learning model parameter until the at least one performance metric satisfies an analytic threshold.

In some embodiments, the LLM routine is a generative pretrained transformer routine that is performed by a generative pretrained transformer, and the generative pretrained transformer is trained by a reinforcement learning model.

In some embodiments, the instructions further include identifying at least one data type of the structured data and receiving an input corresponding to a desired analysis type, and the instructions for performing the large language LLM routine to transform the data into the structured data further include: performing a dimensionality reduction routine based on the data, the at least one data type, and the desired analysis type to generate input data, and performing a generative pretrained transformer routine to transform the input data into the structured data.

The present disclosure provides a system including a processor, a plurality of health databases, where each of the plurality of health databases has a respective syntactic standard, a phenotype routine database that stores a plurality of template phenotype routines, and a nontransitory computer-readable medium including instructions that are executable by the processor. The instructions include receiving data from at least one health database of the plurality of health databases, performing a generative pretrained transformer routine to transform the data into structured data, selecting a template phenotype routine of the plurality of template phenotype routines based on a comparison of at least one performance metric of the selected template phenotype routine and a model selection criteria, generating a phenotype routine by selectively modifying, based on a comparison of the at least one performance metric of and an analytic threshold, at least one parameter of the selected template phenotype routine, analyzing the structured data based on the phenotype routine to generate an output that defines a relationship model associated with the structured data, and transmitting a command to a user interface to generate a display corresponding to the output.

The present disclosure provides a method including receiving data from at least one health database of a plurality of health databases, where each of the plurality of health databases has a respective syntactic standard, performing a generative pretrained transformer routine to transform the data into structured data, selecting a template phenotype routine of a plurality of template phenotype routines based on a comparison of at least one performance metric of the selected template phenotype routine and a model selection criteria, where the plurality of template phenotype routines are stored in a phenotype routine database, generating a phenotype routine by selectively modifying, based on a comparison of the at least one performance metric of and an analytic threshold, at least one parameter of the selected template phenotype routine, analyzing the structured data based on the phenotype routine to generate an output that defines a relationship model associated with the structured data, and transmitting a command to a user interface to generate a display corresponding to the output.

### BRIEF DESCRIPTION OF THE DRAWINGS

In order that the disclosure may be well understood, there will now be described various forms thereof, given by way of example, reference being made to the accompanying drawings, in which:

FIG. 1 illustrates a functional block diagram of a system including a plurality of user computing devices and a computing system in accordance with the teachings of the present disclosure;

FIG. 2 illustrates a functional block diagram of a structured data generator module in accordance with the teachings of the present disclosure;

FIG. 3 illustrates a functional block diagram of a reinforcement learning system that is configured to train a structured data generator module in accordance with the teachings of the present disclosure;

FIG. 4 illustrates a functional block diagram of a phenotype routine generator module in accordance with the teachings of the present disclosure;

FIG. 5 illustrates a functional block diagram of another system including a plurality of user computing devices and a computing system in accordance with the teachings of the present disclosure;

FIG. 6 illustrates an output generated by a test data generator module in accordance with the teachings of the present disclosure; and

FIG. 7 is a flowchart of an example routine for generating and displaying a machine-learning based relationship model in accordance with the teachings of the present disclosure.

The drawings described herein are for illustration purposes only and are not intended to limit the scope of the present disclosure in any way.

### DETAILED DESCRIPTION

The terminology used in the description of the disclosure herein is for the purpose of describing particular embodiments only and is not intended to be limiting of the disclosure. It should be understood that throughout the drawings, corresponding reference numerals indicate like or corresponding parts and features.

In the figures, the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the phrase at least one of A, B, and C should be construed to mean a logical (A OR B OR C), using a non-exclusive logical OR, and should not be construed to mean “at least one of A, at least one of B, and at least one of C.”

In this application, the term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality, such as, but not limited to, transceivers, routers, input/output interface hardware, among others; or a combination of some or all of the above, such as in a system-on-chip. The term “code,” as used herein, may include software, firmware, and/or microcode, and may refer to computer programs, routines, functions, classes, data structures, and/or objects. The computer programs may include: (i) descriptive text to be parsed, (ii) assembly code, (iii) object code generated from source code by a compiler, (iv) source code for execution by an interpreter, (v) source code for compilation and execution by a just-in-time compiler, etc. The term “memory” is a subset of the term computer-readable medium. The term “nontransitory computer-readable medium,” as used herein, does not encompass transitory electrical or electromagnetic signals propagating through a medium (such as on a carrier wave). Non-limiting examples of a non-transitory, tangible computer-readable medium are nonvolatile memory circuits, such as a flash memory circuit, an erasable programmable read-only memory circuit, or a mask read-only circuit, and volatile memory circuits, such as a static random access memory circuit or a dynamic random access memory circuit.

The present disclosure is described herein with reference to flowchart and/or block diagram illustrations of methods, systems, and devices in accordance with exemplary embodiments of the present disclosure. It will be understood that each block of the flowchart and/or block diagram illustrations, and combinations of blocks in the flowchart and/or block diagram illustrations, may be implemented by computer program instructions and/or hardware operations. These computer program instructions may be provided to a processor of a general purpose computer, a special purpose computer, or other programmable data processing apparatus to produce a machine, such that the instructions, which executed via the processor of the computer or other programmable data processing apparatus, implement the functions specified in the flowchart and/or block diagram block or blocks.

The present disclosure provides a computing system that includes a plurality of distinct machine learning and/or artificial intelligence systems (e.g., an ensemble machine learning system) that collectively operate to obtain and process relevant health data and employ an accurate model for analyzing the processed health data. Specifically, the computing system employs a generative pretrained transformer (GPT) module to transform biomedical literature, clinical trial records, observational study reports, regulatory updates, published content, and/or other types of health data into structured data that is suitable for downstream analysis by an additional machine learning system. Moreover, one or more users of remote computing devices coupled to the computing system may select and customize the additional machine learning system (e.g., a supervised learning model, an unsupervised learning model, or a reinforcement learning model) based on one or more corresponding performance metrics of multiple machine learning systems.

The computing system described herein, which integrates the GPT with a customizable machine learning system, provides several improvements to conventional computing systems that obtain, process, and analyze health data for medical-based research, such as clinical and observational studies. As an example, the GPT described herein transforms the health data into structured data that identifies, for example, various medical entities (e.g., diseases, genes, symptoms, medications, and procedures) present in unstructured text data and determines relationships among the unstructured data. As another example, the GPT described herein reduces the dimensions of the feature vectors provided to the selected/customized machine learning system, thereby enabling the computing system to operate with decreased memory and computational resources and improved speed and accuracy.

Accordingly, the transformation routines performed by the GPT enable a separate machine learning system to accurately and more efficiently analyze the data to identify causal pathways, patterns, trends, relationships, and/or predictive models for improving patient care, improving clinical decision-making, predicting clinical trial success rates, predicting responses to treatments, predicting potential adverse events, identifying associations between exposures and diseases, identifying connections between drugs, among other implementations. Moreover, the transformation routines performed by the GPT enable the computing system to produce tailored summaries, provide explanations for predictive models, or amalgamate disjointed pieces of health data to generate new structured data to be analyzed by the separate machine learning system.

Additionally, the computing system described herein employs contextual information identified by a retrieval augmented generation (RAG) routine to further improve the accuracy of the structured data generated by the GPT. The contextual information enables the computing system to more accurately identify relevant medical entities and relationships therebetween based on the input health data, thereby enhancing regulatory guidance review in pharmacovigilance and drug safety. That is, the contextual information enables the computing system to analyze relevant regulatory documents from global health authorities and produce accurate safety reports and compliance documents. The contextual information ensures real-time alignment with the latest regulations and precise insights during safety assessments and adverse event reporting. In some embodiments, the RAG routines enables the computing system to adapt to complex scenarios by incorporating external knowledge, improving the compliance and quality of evaluations, and proactively updating users with changes in regulatory guidelines to ensure that the structured data is current and compliant.

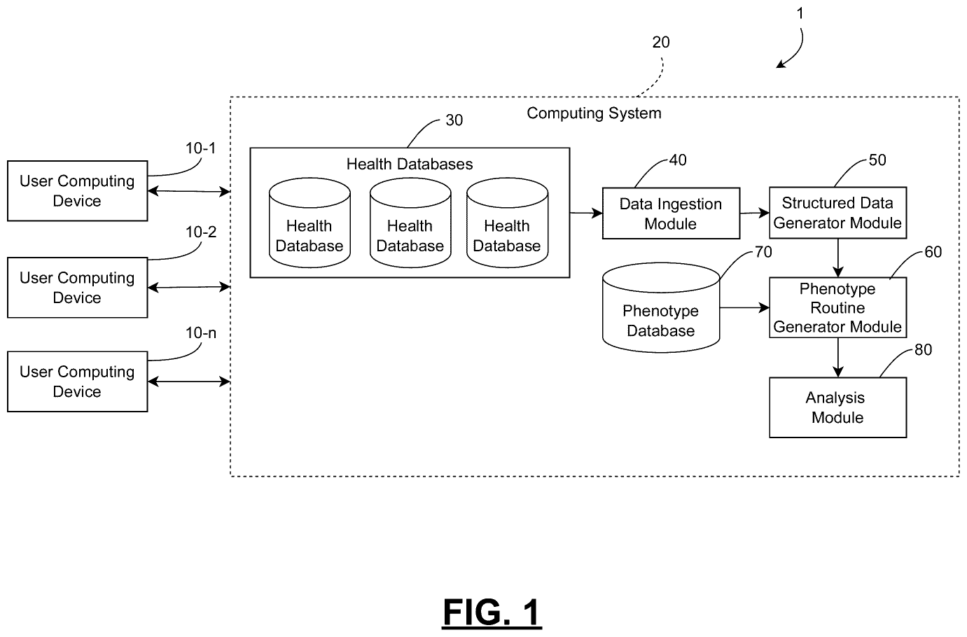

Referring to FIG. 1, a system 1 is provided and generally includes user computing devices 10-1, 10-2, . . . 10-n (collectively referred to hereinafter as “user computing devices 10”) and a computing system 20. It should be readily understood that any one of the components of the system 1 can be provided at the same location or distributed at different locations (e.g., via one or more edge computing devices) and coupled accordingly. In one embodiment, the components of the system 1 are coupled using known wired communication and/or wireless communication protocols (e.g., a cellular protocol, a wireless fidelity (Wi-Fi) protocol, etc.).

In some embodiments, the computing system 20 may include a plurality of health databases 30, a data ingestion module 40, a structured data generator module 50, a phenotype routine generator module 60, a phenotype database 70, and an analysis module 80. To perform the functionality described herein, each of the user computing devices 10 and the computing system 20 may include one or more nontransitory computer-readable mediums and one or more processors that are configured to execute instructions stored in the one or more nontransitory computer-readable mediums.

The user computing devices 10 may include several known computing devices, such as a desktop computer, laptop, smartphone, and/or wearable device. Each of the user computing devices 10 may be operated and controlled by an authorized or registered user of the computing system 20. The user computing devices 10 may include a user interface that enables a user to perform various functions described below in further detail. As an example, the user computing devices 10 may include various display and input devices that collectively enable a user to generate inputs corresponding to the selection of health data from the health databases 30, generate phenotype routines based on a desired analysis type and the type of data, analyze the data using the generated phenotype routine to generate a relationship model associated with the data, and/or visualize the relationship model. As another example, the user computing devices 10 may enable users to modify parameters of phenotype routines generated by other user computing devices and/or modify data retrieved by other user computing devices 10.

In some embodiments, the plurality of health databases 30 are configured to store health data. As an example, the health data may include, but is not limited to, patient population information, patient narratives, provider notes, diagnoses, procedures, medication and/or drug data, lab results, medical literature, clinical trial records, observational studies, regulatory guidelines, research articles, scientific papers, open source code, medical literature, and/or any other types of health data.

In some embodiments, each of the plurality of health databases 30 has a respective syntactic standard. As used herein, “syntactic standard” refers to the structure and syntax of the stored data. As an example, the syntactic standards may include known syntactic standards including, but not limited to, Systematized Nomenclature of Medicine-CPT (Current Procedural Terminology), HCPCS (Healthcare Common Procedure Coding System), ICD-9-CM (International Classification of Diseases, 9th Revision, Clinical Modification), Clinical Terms (SNOMED CT), Logical Observation Identifiers, Names and Codes (LOINC), RxNorm, Concept Unique Identifier (RxCUI), ICD-10-CM (International Classification of Diseases, 10th Revision, Clinical Modification), and ICD-10-PCS (International Classification of Diseases, 10th Revision, Procedure Coding System).

In some embodiments, the plurality of health databases 30 are syntactically and semantically interoperable (i.e., configured to transmit/receive data therebetween and accurately interpret data communicated therebetween) due to one or more defined mapping relationships and application programming interfaces (APIs) therebetween. As an example, the mapping relationships may be defined based on an ontology mapping routine, a schema mapping routine, a crosswalk and code mapping routine, among other known mapping routines. In some embodiments, known software tools may be utilized to define the mapping relationships among the plurality of health databases 30.

The data ingestion module 40 is configured to receive the health data from the plurality of health databases 30 based on, for example, one or more inputs received via the user computing devices 10 that indicate at least one data type (e.g., health data indicating oral contraceptive usage and obesity) and a desired analysis type (e.g., a correlation between oral contraceptive usage and obesity). In some embodiments, the data ingestion module 40 is configured to perform various data processing routines on the health data to improve the accuracy and efficiency of the operation of the structured data generator module 50. Example data processing routines include, but are not limited to, aggregation routines (e.g., combining multiple data elements of the health data into a single item/data element), attribute modification routines (e.g., removing portions of the health data that are irrelevant or have a negligible effect on the output of the structured data generator module 50), and/or data supplement routines (e.g., interpolation or averaging of at least a set of elements of the health data to estimate the values of missing data elements of the health data).

In some embodiments, the data ingestion module 40 is configured to perform a retrieval augmented generation (RAG) routine (e.g., a dense retrieval routine and/or a sparse retrieval routine) to identify contextual information from the plurality of health databases 30 or other external/validated databases associated with the health data. As described below in further detail, the structured data generator module 50 may embed the contextual information with the health data to thereby improve the accuracy of the generated structured data.

In some embodiments, the structured data generator module 50 is configured to transform the health data received by the data ingestion module 40 into structured data. As used herein, “structured data” refers to data having a uniform syntax and type despite the variations of the syntactic standards of the received health data (e.g., both structured and unstructured data). As an example, the structured data generator module 50 may be a natural language processing (NLP) module that is configured to perform an NLP routine (e.g., tokenization, stop word removal, stemming, lemmatization, part-of-speech tagging, and entity recognition routines) to extract relevant features from the varying syntactic representations of the health data and transform it into the structured data having the uniform syntax and type (e.g., a feature vector having a predetermined number of dimensions). As another example, the structured data generator module 50 may be configured to perform functional programming routines to transform the health data into the structured data.

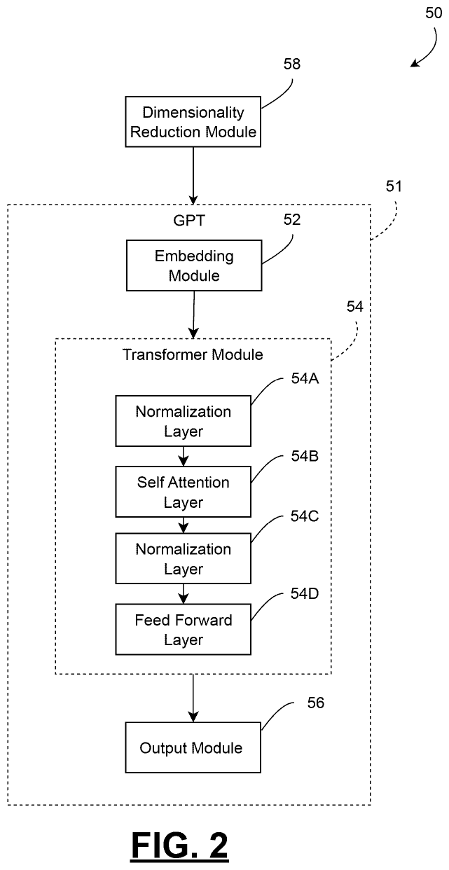

As another example, the structured data generator module 50 may include a generative pretrained transformer (GPT), such as a generative pretrained transformer 2 (GPT-2) or a generative pretrained transformer 3 (GPT-3). As a specific example, and referring to FIGS. 1-2, the structured data generator module 50 may include a GPT 51 and a dimensionality reduction module 58. The GPT 51 may include an embedding module 52, a transformer module 54, and an output module 56. The embedding module 52 is configured to tokenize the health data and embed each of the generated tokens with positional information relative to other generated tokens. A self-attention layer 54B of the transformer module 54 is configured to capture different relationships by generating attention scores associated with the tokens, performing a SoftMax routine to normalize the attention scores into a probability distribution, and outputting a weighted sum for each token to capture the relationships and dependencies associated with the tokens. Accordingly, the embedding module 52 and the self-attention layer 54B are collectively configured to analyze the syntactic structure of the health data to identify, based in part on the contextual information, relevant medical entities among the health data (e.g., diseases, genes, symptoms, medications, and procedures) and determine relationships among the identified medical entities. A feed forward layer 54D of the transformer module 54 is configured to apply a plurality of linear transformations and a non-linear activation function (e.g., a ReLU) to extract additional features of the tokens that are not extracted by the self-attention layer 54B. In some embodiments, the GPT may include normalization layers 54A, 54C to improve the convergence of the GPT 51 and prevent overfitting and vanishing gradients. Accordingly, the GPT 51 is configured to, for example, scrape the literature for existing terminology, map the identified existing terminology to structured definitions, and present the structured definitions in a manner that enables the users of the user computing devices 10 to design and curate the appropriate variables for a desired analysis and/or study. That is, the combination of tokenization, self-attention, and feed-forward transformation routines described herein enables the GPT 51 to process complex medical information and extract meaningful insights associated with the medical information.

In some embodiments, the dimensionality reduction module 58 of the structured data generator module 50 is configured to reduce the number of dimensions of the structured data to a predetermined number. As an example, the dimensionality reduction module 58 is configured to perform a principal component analysis (PCA) routine, a linear discriminant analysis (LDA) routine, an independent component analysis (ICA) routine, a gaussian random projection routine, among other known dimensionality reduction routines to reduce the number of dimensions of the structured data based on the data type(s) of the structured data and/or the desired analysis type. Reducing the dimensions of the structured data may reduce the memory and processor-based computing resources that are implemented for analyzing the structured data, as described below in further detail. Furthermore, reducing the dimensions of the structured data prevents the generated phenotype routine, which is described below in further detail, from exhibiting inaccuracies due to model overfitting.

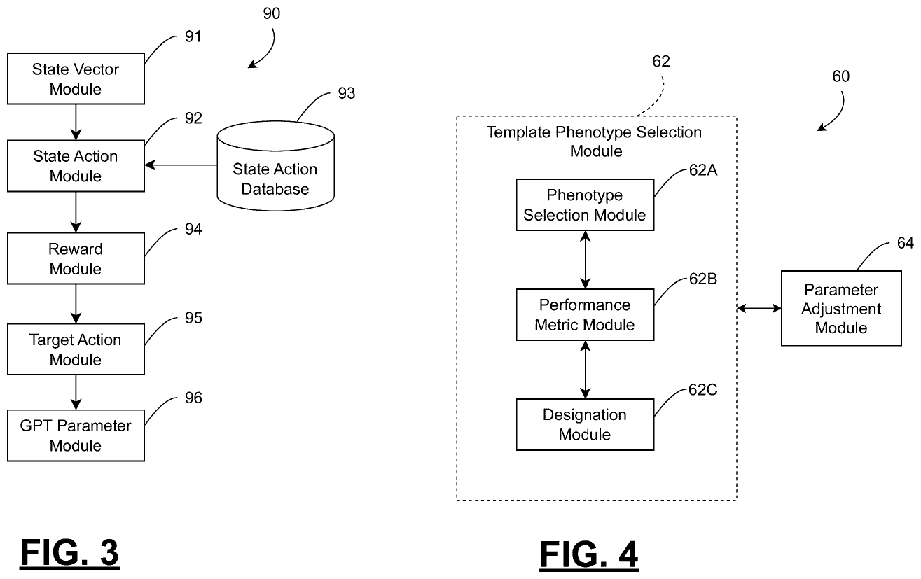

While an example GPT 51 is described herein, it should be understood that the GPT 51 may include additional (or fewer) components that are not explicitly described herein. It should also be understood that the GPT 51 may be trained using known supervised training routines and using various types and/or amounts of health data from the health databases 30. As an example, and referring to FIGS. 1-3, the GPT 51 may be trained by a reinforcement learning model 90, which includes a state vector module 91, a state action module 92, a state action database 93, a reward module 94, a target action module 95, and a GPT parameter module 96. The state vector module 91 may obtain outputs generated by the GPT 51 during a training routine and may generate a plurality of state vectors based on the outputs. Each state vector may correspond to a unique combination of health data received by the data ingestion module 40 from the plurality of health databases 30. The state action module 92 is configured to identify a set of actions from among a plurality of actions stored in the state action database 93 associated with state vectors. The actions may include, but are not limited to, modifying a parameter of the embedding module 52 (e.g., adjusting the dimensionality of the positional embeddings), modifying a parameter of at least one of the normalization layers 54A, 54C, modifying a parameter of the self-attention layer 54B (e.g., adjusting the scaling of the attention scores, adjusting the weights of the SoftMax function, etc.), modifying a parameter of the feed forward layer 54D (adjusting a bias vector and/or weight matrix of at least one of the linear transformations, adjusting the weights of the activation function, etc.), or a combination thereof. In some embodiments, each entry of the state action database 93 is associated with a given action or combination of actions.

The reward module 94 is configured to determine a reward for each action from among the set of actions stored in the state action database 93 using known reinforcement routines. The reward value may be indicative of a qualitative or quantitative metric associated with the predicted resulting accuracy (or improvement) to the GPT 51 based on a selected action (or set of actions). As an example, larger reward values may correspond to improvements in the accuracy in which the GPT 51 generates textual outputs having characteristics that match the desired characteristics of structured data, such as a desired syntax and/or other character-based formatting. As another example, lower reward values may correspond to reductions (or negligible increases) in the accuracy in which the GPT 51 generates textual outputs having characteristics that match the desired characteristics of structured data. In some embodiments, the reward value may be further based on an input received from the user computing devices 10 indicating a predicted resulting accuracy associated with the selected action. The target action module 95 selects a given action from among the set of actions based on each of the awards (e.g., the given action has may have largest or greatest reward value) and instructs the GPT parameter module 96 to modify one or more parameters of the GPT 51 based on the corresponding actions.

When the GPT 51 is sufficiently trained, the output module 56 is configured to output structured data (i.e., data having a uniform syntax and type despite the variations of the syntactic standards of the received health data). As an example, when the health data includes qualitative data (e.g., gender percentage, ethnicity, population size, medications, etc.) and unstructured data (e.g., patient narratives, clinician notes, etc.), the output module 56 is configured to output, based on the GPT routine performed by the GPT 51, structured data (e.g., a feature vector having various rows, columns, and text-based variables therein) that is representative of the obtained qualitative and unstructured data.

Referring to FIGS. 1 and 4, the phenotype routine generator module 60 may include a template phenotype routine selection module 62 and a parameter adjustment module 64. The template phenotype routine selection module 62 may include a phenotype selection module 62A, a performance metric module 62B, and a designation module 62C. The phenotype selection module 62A is configured to select a template phenotype routine from among a plurality of template phenotype routines that are validated and stored in the phenotype database 70 based on an input from the user computing device indicating the type(s) of health data and/or the desired analysis type. As used herein, “phenotype routine” refers to a machine learning and/or artificial intelligence model that is configured to generate a relationship model associated with the structured data. As an example, the phenotype routines may include various types supervised learning models, such as linear regression models, logistic regression models, polynomial regression models, classification models, decision tree models, support vector machine models, Bayesian network models, Gaussian models, discriminant analysis models, neural network models, ensemble models employing a combination of various types of supervised learning models, etc. As another example, the phenotype routines may include unsupervised models, such as clustering models (e.g., k-means clustering and/or hierarchical clustering models), dimensionality reduction models, self-organizing map (SOM) models, etc. As yet another example, the phenotype routines may include reinforcement learning models, such as value-based models, Q-learning models, etc.

The performance metric module 62B is configured to determine at least one performance metric of the selected template phenotype routine. In some embodiments, the performance metric is a value that indicates a capability of the selected template phenotype routine to accurately perform the respective routine (e.g., a classification routine, a clustering routine, etc.).

As an example, the performance metric may include at least one of a positive predictive value (PPV), a negative predictive value (NPV), a sensitivity value, and a specificity value of the selected template phenotype routine. As used herein, “PPV” is a value that indicates a proportion of correct positive predictions relative to all correct and false positive predictions made by the selected template phenotype routine, and “NPV” is a value that indicates a proportion of correct negative predictions relative to all correct and false negative predictions made by the selected template phenotype routine. Higher PPVs and NPVs may be associated with relatively higher accuracies of the selected template phenotype routine. As used herein, “sensitivity value” is a value that indicates a proportion of correct positive predictions relative to all correct positive predictions and all false negative predictions made by the selected template phenotype routine. The “specificity value” is a value that indicates a proportion of correct negative predictions relative to a sum of all correct negative predictions and all false positive predictions made by the selected template phenotype routine.

As another example, the performance metric may include various parameters of a reinforcement learning model, such as a function weight, a learning rate, a discount factor, a replay buffer size, a batch size, normalization parameters, reward functions, action space, and/or state space representations. It should be understood that the performance metric may be any type of value indicating the capability of the selected template phenotype routine to accurately perform the respective routine and is not limited to the example performance metrics described herein.

The designation module 62C is configured to designate the selected template phenotype routine as the phenotype routine when the at least one performance metric satisfies the model selection criteria. In some embodiments, the model selection criteria may correspond to threshold values for at least one of the performance metrics, and the model selection criteria may be satisfied when the at least one performance metric is greater than the threshold value. When the model selection criteria is not satisfied, the phenotype selection module 62A, the performance metric module 62B, and the designation module 62C may iteratively perform the functionality described herein until an additional (e.g., a second) template phenotype routine satisfies the model selection criteria.

As an example, the phenotype selection module 62A may initially identify, based on one or more inputs received via the user computing devices 10, a supervised learning model from the phenotype database 70, such as a neural network model. The performance metric module 62B may then determine at least one performance metric of the neural network model based on the structured data, such as a PPV, a NPV, a sensitivity value, and/or a specificity value and determine whether the at least one performance metric of the neural network model satisfies the model selection criteria. For example, the at least one performance metric value may satisfy the model selection criteria when each of the PPV is greater than a first threshold PPV, the NPV is greater than a first threshold NPV, the sensitivity value is greater than a first threshold sensitivity value, and the specificity value is greater than a first threshold specificity value. As another example, the at least one performance metric value may satisfy the model selection criteria when at least one of the PPV is greater than the first threshold PPV, the NPV is greater than the first threshold NPV, the sensitivity value is greater than the first threshold sensitivity value, and the specificity value is greater than the first threshold specificity value. The designation module 62C may then designate the neural network model as the selected template phenotype routine in response to a determination that the at least one performance metric value satisfies the model selection criteria. Otherwise, the designation module 62C may not designate the neural network model as the selected template phenotype routine in response to a determination that the at least one performance metric does not satisfy the model selection criteria. The phenotype routine generator module 60 may iteratively perform the functionality described herein until it identifies a template supervised, unsupervised, or reinforcement learning model from the phenotype database 70 having at least one performance metric that satisfies the model selection criteria.

In response to the template phenotype routine selection module 62 selecting the template phenotype routine, the parameter adjustment module 64 is configured to generate a phenotype routine by selectively modifying at least one parameter of the selected template phenotype routine. In some embodiments, the parameter adjustment module 64 is configured to iteratively modify one or more parameters of the selected template phenotype routine until the at least one performance metric satisfies one or more analytic thresholds.

As an example, the template phenotype routine selection module 62 may designate the neural network model as the selected template phenotype routine due to each of the PPV being greater than a first threshold PPV, the NPV being greater than a first threshold NPV, the sensitivity value being greater than a first threshold sensitivity value, and the specificity value being greater than a first threshold specificity value. Subsequently, the parameter adjustment module 64 may iteratively modify one or more parameters of the neural network model (e.g., a number of dimensions of the structured data, weights, biases, number of layers and neurons per layer, types of activation functions, etc.) until the at least one performance metric (e.g., the PPV, the NPV, the sensitivity value, and/or the specificity value) satisfies the analytic threshold. For example, the at least one performance metric value may satisfy the analytic threshold when at least one of the PPV is greater than a second threshold PPV that is different from (e.g., greater) the first threshold PPV, the NPV is greater than a second threshold NPV that is different from (e.g., greater) the first threshold NPV, the sensitivity value is greater than a second threshold sensitivity value that is different from (e.g., greater) the first threshold sensitivity value, and the specificity value is greater than a second threshold specificity value that is different from (e.g., greater) the first threshold specificity value.

As another example, the template phenotype routine selection module 62 may designate a clustering model as the selected template phenotype routine due to at least one of the PPV being greater than a first threshold PPV, the NPV being greater than a first threshold NPV, the sensitivity value being greater than a first threshold sensitivity value, and the specificity value being greater than a first threshold specificity value. Subsequently, the parameter adjustment module 64 may iteratively modify one or more parameters of the clustering model, such as a cluster characteristic (e.g., a number of dimensions of the selected template phenotype routine, a number of clusters of the selected template phenotype routine, and/or a distance metric of the selected template phenotype routine, etc.) until the at least one performance metric (e.g., the PPV, the NPV, the sensitivity value, and/or the specificity value) satisfies one or more analytic thresholds, as described above.

As an additional example, the template phenotype routine selection module 62 may designate a reinforcement learning model as the selected template phenotype routine due to at least one of the PPV being greater than a first threshold PPV, the NPV being greater than a first threshold NPV, the sensitivity value being greater than a first threshold sensitivity value, and the specificity value being greater than a first threshold specificity value. Subsequently, the parameter adjustment module 64 may iteratively modify one or more reinforcement parameters of the reinforcement model (e.g., a function weight, a learning rate, a discount factor, a replay buffer size, a batch size, normalization parameters, reward functions, action space, and/or state space representations) until the at least one performance metric (e.g., the PPV, the NPV, the sensitivity value, and/or the specificity value) satisfies one or more analytic thresholds, as described above.

The analysis module 80 is configured to analyze the structured data based on the phenotype routine to generate an output that defines a relationship model associated with the structured data and transmit a command to a user interface of the user computing devices 10 corresponding to the output. As used herein, “relationship model” refers to any mathematical, visual, or other model type that can be output by the phenotype routine (e.g., a machine learning or artificial intelligence model) indicating a relationship among the structured data. As an example, when the phenotype routine is a linear regression model, the relationship model associated with the structured data may be a mathematical predictive model and/or graphical representation configured to predict one or more outputs based on the structured data. As another example, when the phenotype routine is a clustering model, the relationship model associated with the structured data may be a graphical representation of a plurality of multidimensional data points, the corresponding clusters, and the cluster characteristics.

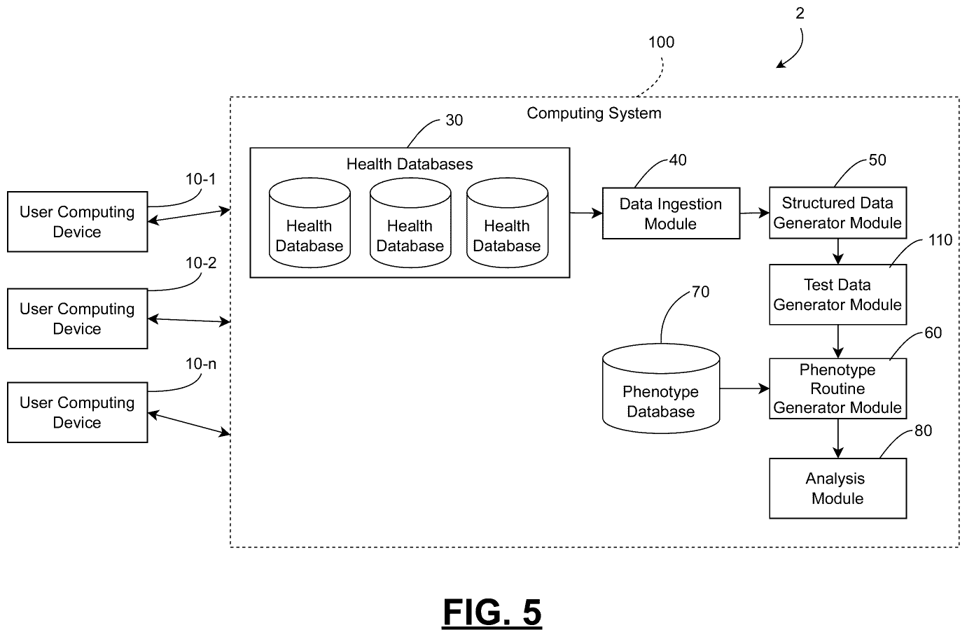

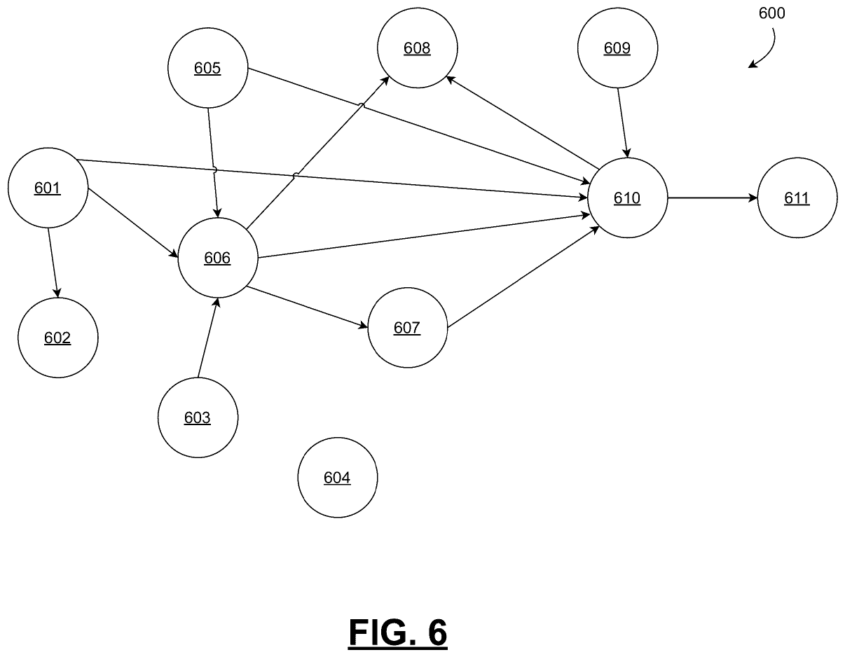

Referring to FIG. 5, a system 2 is provided and includes the user computing devices 10 and a computing system 100, which is similar to the computing system 20 illustrated in FIG. 1 except that it further includes a test data generator module 110. In some embodiments, the test data generator module 110 is configured to generate test data based on the structured data (and optionally, the contextual information output by the data ingestion module 40 to enhance the test data and/or structured data, such as a causal diagram) for downstream analysis by the analysis module 80. As an example and referring to FIGS. 5-6, the test data generator module 110 is configured to generate a directed acyclic graph (DAG) 600 based on the structured data generated by the structured data generator module 50. In some embodiments, the DAG 600 includes nodes 601, 602, 603, 604, 605, 606, 607, 608, 609, 610, 611 (collectively referred to hereinafter as “the nodes”) that each represent a variable or portion of the structured data (e.g., a medical entity, such as diabetic retinopathy, hypertension, age, medication type, outcomes, risk factors, noise variables, etc.) and at least one causal relationship (shown by the arrows/directed edges of the DAG 600). It should be understood that the DAG 600 may include any number of nodes and causal relationships therebetween in other embodiments and is not limited to the example illustrated and described herein. In some embodiments, the nodes and/or causal relationships may be selectively modified based on inputs received from additional or other users of the user computing devices 10 (e.g., the DAG 600 is collaboratively defined by several users of the respective user computing devices 10).

As an example, the test data generator module 110 generates the nodes of the DAG 600 based on the medical entities identified by the embedding module 52 and the self-attention layer 54B of the GPT 51 and generates the causal relationships based on the relationships among the identified medical entities identified by the embedding module 52 and the self-attention layer 54B. Subsequently, the test data generator module 110 is configured to transform the DAG 600 into test data having the same syntax as the structured data using known graph to text-based syntax (e.g., R code) conversion software products, such as DAGitty®. Thus, the phenotype routine executed by the analysis module 80 for further analysis may employ the test data, which may extract additional relationships among the identified entities/variables of the structured data as opposed to solely employing the structured data.

Accordingly, the analysis module 80 may analyze the DAG 600 to estimate a causal effect of an exposure on an outcome. As an example, the analysis module 80 may identify confounding paths, which refer to open paths between the exposure and outcome that pass through one or more confounders. These confounding paths may introduce bias, which is mitigated by conditioning one or more nodes of the DAG 600 along an adjacent path and by designating the one or more nodes as covariates. As another example, the analysis module 80 may analyze the DAG 600 to identify collider paths, which refer to closed paths between the exposure and outcome that pass through one or more colliders. Colliders refer to nodes that receive multiple incoming arcs from the exposure and outcome. The analysis module 80 may determine that collider paths do not transmit associations unless colliders or their descendants are conditioned, which introduces collider bias. Moreover, the analysis module 80 accounts for scenarios where conditioning a mediator, which may be influenced by common causes of the outcome, may introduce collider bias, as the mediator also functions as a collider. As yet another example, the analysis module 80 determines a sufficient adjustment set for the causal estimation. The sufficient adjustment set comprises a set of variables that, when conditioned, provides an unbiased estimate by closing all non-causal paths and leaving causal paths open. The analysis module 80 may further generate competing exposures in the adjustment set when these exposures act as surrogate confounders caused by unobserved confounders.

Moreover, employing contextual information generated by the data ingestion module 40 via the RAG routine may further streamline the efficacy in which covariates, mediators, confounders, colliders, and other variables are identified when the analysis module 80 identifies causal estimands associated with the identified entities/variables of the DAG 600. For example, the contextual information enables the test data generator module 110 to generate new nodes and identify new relationships of the DAG 600, thereby ensuring a more accurate and comprehensive representation of causal pathways. Additionally, the contextual information enables the analysis module 80 to identify confounders, mitigate bias, and generate recommended adjustments by considering insights from the contextual information. For regulated fields like pharmacovigilance and drug safety, the contextual information ensures that the DAG 600 is aligned with the latest regulatory standards, improving both compliance and precision in decision-making by the analysis module 80.

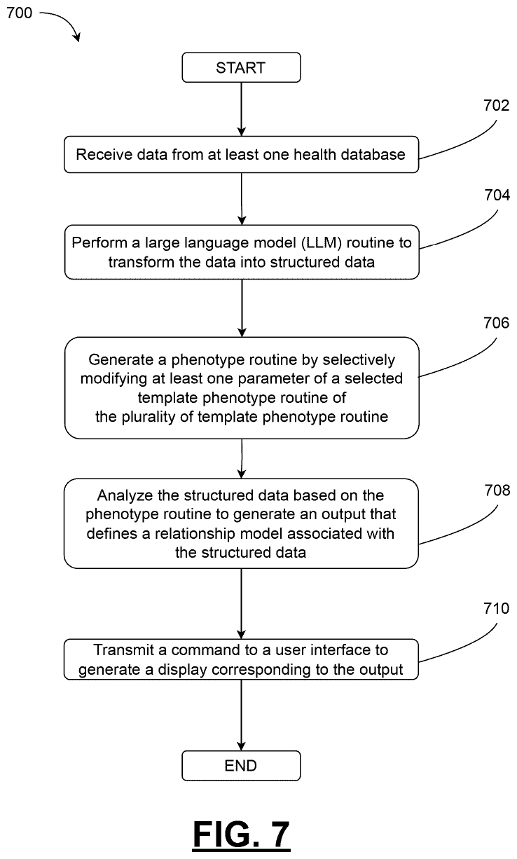

Referring to FIG. 7, a flowchart 700 of an example routine for generating and displaying a machine-learning based relationship model is shown. At 702, the data ingestion module 40 receives data (e.g., health data) from the at least one health database 30. At 704, the structured data generator module 50 performs a large language model routine (e.g., a GPT routine) to transform the data into structured data. At 706, the phenotype routine generator module 60 generates a phenotype routine by selectively modifying at least one parameter of a selected template phenotype routine from the phenotype database 70. At 708, the analysis module 80 analyzes the structured data based on the phenotype routine to generate an output that defines a relationship model associated with the structured data. At 710, the analysis module 80 transmits a command to a user interface (e.g., a user interface of the user computing device 10, such as display) to generate a display corresponding to the output.

The present disclosure is described with reference to the accompanying drawings, in which embodiments of the disclosure are shown. However, this disclosure should not be construed as limited to the embodiments set forth herein. Rather, these embodiments are provided so that this disclosure will be thorough and complete, and will fully convey the scope of the disclosure to those skilled in the art. Thus, variations that do not depart from the substance of the disclosure are intended to be within the scope of the disclosure. Such variations are not to be regarded as a departure from the scope of the disclosure. Accordingly, all such variations are intended to be included within the scope of example embodiments as defined in the claims.

What is claimed is:

1. A system comprising:
a processor;
a plurality of health databases, wherein each of the plurality of health databases has a respective syntactic standard;
a phenotype routine database that stores a plurality of template phenotype routines; and
a nontransitory computer-readable medium comprising instructions that are executable by the processor, wherein the instructions comprise:
receiving data from at least one health database of the plurality of health databases;
performing a large language model (LLM) routine to transform the data into structured data;
generating a phenotype routine by selectively modifying at least one parameter of a selected template phenotype routine of the plurality of template phenotype routines;
analyzing the structured data based on the phenotype routine to generate an output that defines a relationship model associated with the structured data; and
transmitting a command to a user interface to generate a display corresponding to the output.

2. The system of claim 1, wherein the LLM routine is one of a generative pretrained transformer routine or a natural language processing routine.

3. The system of claim 1, wherein the instructions further comprise:
generating a directed acyclic graph based on the structured data and at least one causal relationship associated with the structured data; and
transforming the directed acyclic graph into test data, wherein the test data and the structured data have a same syntax,
wherein analyzing the structured data based on the phenotype routine to generate the output that defines the relationship model associated with the structured data further comprises analyzing the test data based on the phenotype routine.

4. The system of claim 1, wherein the instructions further comprise:
selecting a first template phenotype routine of the plurality of template phenotype routines; and
determining at least one performance metric of the first template phenotype routine, wherein the at least one performance metric of the first template phenotype routine comprises at least one of a positive predictive value (PPV), a negative predictive value (NPV), a sensitivity value, and a specificity value of the first template phenotype routine.

5. The system of claim 4, wherein the instructions further comprise designating the first template phenotype routine as the selected phenotype routine when the at least one performance metric of the first template phenotype routine satisfies a model selection criteria.

6. The system of claim 4, wherein the instructions further comprise:
selecting a second template phenotype routine of the plurality of template phenotype routines when the at least one performance metric of the first template phenotype routine does not satisfy a model selection criteria;
determining at least one performance metric of the second template phenotype routine, wherein the at least one performance metric of the second template phenotype routine comprises at least one of a positive predictive value (PPV), a negative predictive value (NPV), a sensitivity value, and a specificity value of the second template phenotype routine; and
designating the second template phenotype routine as the selected phenotype routine when the at least one performance metric of the second template phenotype routine satisfies the model selection criteria.

7. The system of claim 1, wherein:
the instructions further comprise determining at least one performance metric of the selected template phenotype routine;
the at least one parameter comprises a number of dimensions of the selected template phenotype routine; and
the instructions for generating the phenotype routine by selectively modifying the at least one parameter of the selected template phenotype routine further comprise iteratively reducing the number of dimensions of the selected template phenotype routine until the at least one performance metric satisfies an analytic threshold.

8. The system of claim 1, wherein:
the instructions further comprise determining at least one performance metric of the selected template phenotype routine;
the at least one parameter comprises at least one cluster characteristic of the selected template phenotype routine; and
the instructions for generating the phenotype routine by selectively modifying the at least one parameter of the selected template phenotype routine further comprise iteratively modifying the at least one cluster characteristic of the selected template phenotype routine until the at least one performance metric satisfies an analytic threshold.

9. The system of claim 8, wherein the at least one cluster characteristic comprises at least one of a number of dimensions of the selected template phenotype routine, a number of clusters of the selected template phenotype routine, and a distance metric of the selected template phenotype routine.

10. The system of claim 1, wherein:
the instructions further comprise determining at least one performance metric of the selected template phenotype routine;
the at least one parameter comprises a reinforcement learning model parameter of the selected template phenotype routine;
the instructions for generating the phenotype routine by selectively modifying the at least one parameter of the selected template phenotype routine further comprise iteratively modifying the reinforcement learning model parameter until the at least one performance metric satisfies an analytic threshold.

11. The system of claim 1, wherein:
the LLM routine is a generative pretrained transformer routine that is performed by a generative pretrained transformer; and
the generative pretrained transformer is trained by a reinforcement learning model.

12. The system of claim 1, wherein:
the instructions further comprise identifying at least one data type of the structured data and receiving an input corresponding to a desired analysis type; and
the instructions for performing the large language LLM routine to transform the data into the structured data further comprise:
performing a dimensionality reduction routine based on the data, the at least one data type, and the desired analysis type to generate input data; and
performing a generative pretrained transformer routine to transform the input data into the structured data.

13. A system comprising:
a processor;
a plurality of health databases, wherein each of the plurality of health databases has a respective syntactic standard;
a phenotype routine database that stores a plurality of template phenotype routines; and
a nontransitory computer-readable medium comprising instructions that are executable by the processor, wherein the instructions comprise:
receiving data from at least one health database of the plurality of health databases;
performing a generative pretrained transformer routine to transform the data into structured data;
selecting a template phenotype routine of the plurality of template phenotype routines based on a comparison of at least one performance metric of the selected template phenotype routine and a model selection criteria;
generating a phenotype routine by selectively modifying, based on a comparison of the at least one performance metric of and an analytic threshold, at least one parameter of the selected template phenotype routine;
analyzing the structured data based on the phenotype routine to generate an output that defines a relationship model associated with the structured data; and
transmitting a command to a user interface to generate a display corresponding to the output.

14. The system of claim 13, wherein the instructions further comprise:
generating a directed acyclic graph based on the structured data and at least one causal relationship associated with the structured data; and
transforming the directed acyclic graph into test data, wherein the test data and the structured data have a same syntax,
wherein analyzing the structured data based on the phenotype routine to generate the output that defines the relationship model associated with the structured data further comprises analyzing the test data based on the phenotype routine.

15. The system of claim 13, wherein the at least one performance metric comprises at least one of a positive predictive value (PPV), a negative predictive value (NPV), a sensitivity value, and a specificity value.

16. The system of claim 13, wherein the at least one parameter comprises a number of dimensions of the selected template phenotype routine.

17. The system of claim 13, wherein the at least one parameter comprises at least one of a number of dimensions of the selected template phenotype routine, a number of clusters of the selected template phenotype routine, and a distance metric of the selected template phenotype routine.

18. The system of claim 1, wherein the at least one parameter comprises a transient value and a steady state value of the selected template phenotype routine.

19. A method comprising:
receiving data from at least one health database of a plurality of health databases, wherein each of the plurality of health databases has a respective syntactic standard;
performing a generative pretrained transformer routine to transform the data into structured data;
selecting a template phenotype routine of a plurality of template phenotype routines based on a comparison of at least one performance metric of the selected template phenotype routine and a model selection criteria, wherein the plurality of template phenotype routines are stored in a phenotype routine database;
generating a phenotype routine by selectively modifying, based on a comparison of the at least one performance metric of and an analytic threshold, at least one parameter of the selected template phenotype routine;
analyzing the structured data based on the phenotype routine to generate an output that defines a relationship model associated with the structured data; and
transmitting a command to a user interface to generate a display corresponding to the output.

20. The method of claim 19, wherein the at least one performance metric comprises at least one of a positive predictive value (PPV), a negative predictive value (NPV), a sensitivity value, and a specificity value.
